(19) United States
(12) Patent Application Publication
(10) Pub. No.: US 2022/0065840 A1
(43) Pub. Date: Mar. 3, 2022

(54) METHOD FOR ANALYSIS OF THE POSITIONAL DISTRIBUTION OF FATTY ACID IN PHOSPHATIDYLCHOLINE

(71) Applicant: Shaanxi University of Science & Technology, Xi'an Shaanxi Province (CN)

(72) Inventors: Daoming LI, Xi'an Shaanxi Province (CN); Xiaorong ZHONG, Xi'an Shaanxi Province (CN); Duan ZHOU, Xi'an (CN); Panxue WANG, Xi'an (CN); Ning LIU, Xi'an (CN)

(21) Appl. No.: 17/395,343

(22) Filed: Aug. 5, 2021

(30) Foreign Application Priority Data
Aug. 31, 2020 (CN) 202010900910.0

(51) Int. Cl. G01N 33/28; G01N 1/44; G01N 1/38; C12N 9/20

(52) U.S. Cl. CPC G01N 33/28; G01N 1/44; G01N 2030/025; C12N 9/20; G01N 1/38

(57) ABSTRACT

The present disclosure provides a method for analysis of the position distribution of lecithin fat acid, relating to the technical field of oil processing. The analysis method according to the present disclosure makes it possible to catalyze lecithin to complete alcoholysis in a short time with Novozym 435 or Lipozyme 435 in the present of excess anhydrous ethanol, thereby quickly and accurately analyzing the position distribution of lecithin fat acid. Novozym 435 or Lipozyme 435 exhibits a strong sn-1 position specificity and an extremely high reactivity to lecithin in the present of excess anhydrous ethanol, thereby greatly increasing the reaction rate of the alcoholysis to ensure quick and complete alcoholysis of lecithin, avoiding the occurrence of the transfer of acyl group, and improving the accuracy of the analysis results. Also, the use of the anhydrous ethanol could effectively avoid the generation of fatty acid as a by-product of hydrolysis, and simplify subsequent analysis steps. The method according to the present disclosure has a short analysis time, a simple operation, an accurate measuring result, and a wide range of application.

### TECHNICAL FIELD

The present disclosure relates to the technical field of oil processing, and in particular to a method for analysis of the positional distribution of fatty acid in phosphatidylcholine.

### BACKGROUND ART

Phosphatidylcholine (PC), also known as lecithin, has been recognized as “the third nutrient”, which is parallel to protein and vitamin. It not only has physiological functions such as regulating body metabolism and blood lipid, and improving brain vitality and memory, but also has physics and chemistry functions such as emulsification, release, wetting, anti-oxidation and foaming. PC has a broad application prospect in the fields of food, cosmetics and medicine because of its wide range of functional characteristics. The property of PC mainly depends on the composition of fatty acid therein, and the change of it reflects important differences in metabolism and function. Thus, the molecular structure of PC has an important effect on the uptake rate and total intake thereof. Moreover, better understanding of the positional distribution of fatty acid in PC may provide further information for the research of the biological function of PC. Therefore, the development of the analysis method of the positional distribution of fatty acid in PC has been widely concerned, and the research on the method has become a hotspot.

Enzymatic hydrolysis is a main method for analysis of the positional distribution of fatty acid in PC. It is mainly performed by the specific hydrolysis and alcoholysis of ester bond at sn-1 or sn-2 position of PC through lipase or phospholipase A₂. The enzymatic hydrolysis is mainly divided into single enzymatic hydrolysis and double enzymatic hydrolysis, wherein the double enzymatic hydrolysis needs to use two different enzymes in two reaction systems to measure the composition of fatty acid at sn-1 and sn-2 positions of PC, respectively, which is complex for analysis and operation, and has low accuracy (J. Am. Oil. Chem. Soc., 2004, 81: 553-557). The single enzymatic hydrolysis is simpler for operation than the double enzymatic hydrolysis. In related studies, the single enzymatic hydrolysis only needs to use one sn-1,3 position specific lipases to catalyze the alcoholysis of PC with 95% ethanol, in which the resulting product is separated out by liquid-liquid extraction, and then the separated product is subjected to a methylesterification, to obtain the measuring result of the positional distribution of fatty acid in phosphatidylcholine. The reaction system used for analysis in the single enzymatic hydrolysis is simple; however, the ethanol used therein is 95% ethanol, and the reaction product contains free fatty acids, which increase the complexity of the analysis procedures and reduce the accuracy of the analysis results; moreover, the single enzymatic hydrolysis has a long reaction time (8 h for the complete ethanolification), which increases the risk of the acyl migration of the reaction intermediate product sn2-LPC (Lysophosphatidylcholine) and reduces the accuracy of the analysis results (Food Chem., 2012, 135: 2542-2548). In short, the analysis of the positional distribution of fatty acid in PC by current enzymatic hydrolysis has a long reaction time, complex procedures and a low analysis accuracy.

### SUMMARY

In order to address the above drawbacks in the prior art, the present disclosure provides a method for analysis of the positional distribution of fatty acid in phosphatidylcholine. The method provides an accurate measuring result with short analysis time and simple operation, having a wide range of application.

The present disclosure is realized by the following technical solutions:

A method for analysis of the positional distribution of fatty acid in phosphatidylcholine, comprising,
Step 1: mixing phosphatidylcholine and excess anhydrous ethanol to be uniform in a reaction vessel, to obtain a mixture, heating the mixture to a temperature for an alcoholysis reaction, and connecting a reflux device to the reaction vessel;
Step 2: adding immobilized lipase Novozym 435 or Lipozyme 435 into the reaction vessel, and subjecting the resulting mixture to the alcoholysis reaction while stirring, to obtain an alcoholysis product;
Step 3: extracting the alcoholysis product with water and n-hexane in sequence, to obtain an aqueous phase and an n-hexane phase, and collecting the aqueous phase and the n-hexane phase, respectively; adding cold acetone into the aqueous phase for depositing, to obtain sn2-LPC, and subjecting the n-hexane phase to a rotary evaporation to remove n-hexane, to obtain fatty acid ethyl ester; and
Step 4: directly analyzing the fatty acid ethyl ester by gas chromatography; subjecting the sn2-LPC to a methylesterification, and analyzing the sn2-LPC after the methylesterification by gas chromatography.

In some embodiments, a molar ratio of phosphatidylcholine to anhydrous ethanol is in the range of 1:(40-100).

In some embodiments, in step 1, the temperature for an alcoholysis reaction is 25° C. to 40° C.

In some embodiments, in step 2, the immobilized lipase is added in an amount of 6%-15%, based on the total mass of a substrate.

In some embodiments, in step 2, the stirring is performed at a rotation speed of 250-500 rpm.

In some embodiments, subjecting the resulting mixture to the alcoholysis reaction is performed for 1-3 h.

Compared with the prior art, the present disclosure has the following beneficial effects:

The method for analysis of the positional distribution of fatty acid in phosphatidylcholine according to the present disclosure makes it possible to catalyze phosphatidylcholine to complete alcoholysis in a short time with Novozym 435 or Lipozyme 435 in the present of excess anhydrous ethanol, thereby quickly and accurately analyzing the positional distribution of fatty acid in phosphatidylcholine. Novozym 435 or Lipozyme 435 exhibits a strong sn-1 position specificity and an extremely high reactivity to phosphatidylcholine in the present of excess anhydrous ethanol, thereby greatly increasing the rate of the alcoholysis reaction to ensure quick and complete alcoholysis of phosphatidylcholine, avoiding the transfer of acyl group, thereby improving the accuracy of the analysis results. Also, the use of the anhydrous ethanol could effectively avoid the generation of fatty acid as a by-product of hydrolysis, and simplify subsequent analysis steps. The method according to the present disclosure provides an accurate measuring result with short analysis time and simple operation, having a wide range of application.

In some embodiments, a molar ratio of phosphatidylcholine to anhydrous ethanol is in the range of 1:(40-100). Excess anhydrous ethanol makes it possible to ensure Novozym 435 or Lipozyme 435 to exhibit a strong sn-1 position specificity, an extremely high reactivity to phosphatidylcholine, and a good operation stability.

In some embodiments, in step 1, the temperature for the alcoholysis reaction is 25° C. to 40° C., which could effectively avoid the reduction of reaction rate caused by ethanol volatilization, and ensure that Novozym 435 or Lipozyme 435 has a good operation stability.

In some embodiments, the immobilized lipase is added in an amount of 6%-15% of total mass of a substrate, which could ensure that the lipase used exhibits a strong sn-1 position specificity and an extremely high reactivity to phosphatidylcholine in the present of excess anhydrous ethanol, and ensure the economy of the reaction.

In some embodiments, subjecting the resulting mixture to the alcoholysis reaction is performed for 1-3 h, which could not only ensure the complete conversion of PC into sn2-LPC and fatty acid ethyl ester, but also ensure that Novozym 435 or Lipozyme 435 has a good operation stability.

In some embodiments, the stiffing is performed at a rotation speed of 250-500 rpm, which could not only ensure a higher reaction rate, but also better ensure that the lipase used has a particle integrity.

### DETAILED DESCRIPTION OF THE EMBODIMENTS

The present disclosure will be further illustrated in detail below with reference to the specific examples. It should be understood that the examples are to explain rather than limit the present disclosure. Unless otherwise indicated, all percentages refer to mass percentages.

### Example 1

100 g of a mixture of soybean phosphatidylcholine and anhydrous ethanol (a molar ratio of soybean phosphatidylcholine to anhydrous ethanol being 1:100) was added into a 500 mL round-bottomed flask. The mixture was stirred and mixed to be uniform, and then heated to 30° C. After that, a reflux device was connected to the round-bottomed flask. 15 g of Novozym 435 was added thereto, and the resulting mixture was subjected to a reaction for 1 h with magnetic stirring at a rotation speed of 250 rpm, obtaining an immobilized lipase. The immobilized lipase was collected, and evaporated on a rotary evaporator to remove anhydrous ethanol. The composition of the resulting product was analyzed by liquid chromatography. The analysis result showed that the product had no PC, and only contained sn2-LPC and fatty acid ethyl ester, and the molar numbers of sn2-LPC and fatty acid ethyl ester were equal, and were equal to the initial molar number of PC, showing that PC was completely converted into sn2-LPC and fatty acid ethyl ester. The product was extracted with solvent (water and n-hexane in sequence) to separate sn2-LPC and fatty acid ethyl ester, obtaining an aqueous phase and an n-hexane phase. Cold acetone was added into the aqueous phase for depositing, obtaining sn2-LPC, and sn2-LPC was subjected to a methylesterification, and was then analyzed by gas chromatography to determine composition of fatty acid at sn-2 position of phosphatidylcholine. The n-hexane phase was subjected to a rotary evaporation to remove n-hexane, obtaining fatty acid ethyl ester. Fatty acid ethyl ester was analyzed by gas chromatography to determine composition of fatty acid at sn-1 position of phosphatidylcholine.

### Example 2

100 g of a mixture of soybean phosphatidylcholine and anhydrous ethanol (a molar ratio of soybean phosphatidylcholine to anhydrous ethanol being 1:40) was added into a 500 mL round-bottomed flask. The mixture was stirred and mixed to be uniform, and then heated to 25° C. After that, a reflux device was connected to the round-bottomed flask. 10 g of Lipozyme 435 was added thereto, and the resulting mixture was subjected to a reaction for 3 h with magnetic stirring at a rotation speed of 500 rpm, obtaining an immobilized lipase. The immobilized lipase was collected, and evaporated on a rotary evaporator to remove anhydrous ethanol. The composition of the resulting product was analyzed by liquid chromatography. The analysis result showed that the product had no PC, and only contained sn2-LPC and fatty acid ethyl ester, and the molar numbers of sn2-LPC and fatty acid ethyl ester were equal, and were equal to the initial molar number of PC, showing that PC was completely converted into sn2-LPC and fatty acid ethyl ester. The product was extracted with solvent (water and n-hexane in sequence) to separate sn2-LPC and fatty acid ethyl ester, obtaining an aqueous phase and an n-hexane phase. Cold acetone was added into the aqueous phase for depositing, obtaining sn2-LPC, and sn2-LPC was subjected to a methylesterification, and was then analyzed by gas chromatography to determine composition of fatty acid at sn-2 position of phosphatidylcholine. The n-hexane phase was subjected to a rotary evaporation to remove n-hexane, obtaining fatty acid ethyl ester. Fatty acid ethyl ester was analyzed by gas chromatography to determine composition of fatty acid at sn-1 position of phosphatidylcholine.

### Example 3

100 g of a mixture of soybean phosphatidylcholine and anhydrous ethanol (a molar ratio of soybean phosphatidylcholine to anhydrous ethanol being 1:60) was added into a 500 mL round-bottomed flask. The mixture was stirred and mixed to be uniform, and then heated to 40° C. After that, a reflux device was connected to the round-bottomed flask. 6 g of Novozym 435 was added thereto, and the resulting mixture was subjected to a reaction for 3 h with magnetic stirring at a rotation speed of 400 rpm, obtaining an immobilized lipase. The immobilized lipase was collected, and evaporated on a rotary evaporator to remove anhydrous ethanol. The composition of the resulting product was analyzed by liquid chromatography. The analysis result showed that the product had no PC, and only contained sn2-LPC and fatty acid ethyl ester, and the molar numbers of sn2-LPC and fatty acid ethyl ester were equal, and were equal to the initial molar number of PC, showing that PC was completely converted into sn2-LPC and fatty acid ethyl ester. The product was extracted with solvent (water and n-hexane in sequence) to separate sn2-LPC and fatty acid ethyl ester, obtaining an aqueous phase and an n-hexane phase. Cold acetone was added into the aqueous phase for depositing, obtaining sn2-LPC, and sn2-LPC was subjected to a methylesterification, and was then analyzed by gas chromatography to determine composition of fatty acid at sn-2 position of phosphatidylcholine. The n-hexane phase was subjected to a rotary evaporation to remove n-hexane, obtaining fatty acid ethyl ester. Fatty acid ethyl ester was analyzed by gas chromatography to determine composition of fatty acid at sn-1 position of phosphatidylcholine.

### Example 4

100 g of a mixture of egg yolk phosphatidylcholine and anhydrous ethanol (a molar ratio of egg yolk phosphatidylcholine to anhydrous ethanol being 1:80) was added into a 500 mL round-bottomed flask. The mixture was stirred and mixed to be uniform, and then heated to 30° C. After that, a reflux device was connected to the round-bottomed flask. 10 g of Lipozyme 435 was added thereto, and the resulting mixture was subjected to a reaction for 2 h with magnetic stirring at a rotation speed of 350 rpm, obtaining an immobilized lipase. The immobilized lipase was collected, and evaporated on a rotary evaporator to remove anhydrous ethanol. The composition of the resulting product was analyzed by liquid chromatography. The analysis result showed that the product had no PC, and only contained sn2-LPC and fatty acid ethyl ester, and the molar numbers of sn2-LPC and fatty acid ethyl ester were equal, and were equal to the initial molar number of PC, showing that PC was completely converted into sn2-LPC and fatty acid ethyl ester. The product was extracted with solvent (water and n-hexane in sequence) to separate sn2-LPC and fatty acid ethyl ester, obtaining an aqueous phase and an n-hexane phase. Cold acetone was added into the aqueous phase for depositing, obtaining sn2-LPC, and sn2-LPC was subjected to a methylesterification, and was then analyzed by gas chromatography to determine composition of fatty acid at sn-2 position of phosphatidylcholine. The n-hexane phase was subjected to a rotary evaporation to remove n-hexane, obtaining fatty acid ethyl ester. Fatty acid ethyl ester was analyzed by gas chromatography to determine composition of fatty acid at sn-1 position of phosphatidylcholine.

### Example 5

100 g of a mixture of krill phosphatidylcholine and anhydrous ethanol (a molar ratio of krill phosphatidylcholine to anhydrous ethanol being 1:60) was added into a 500 mL round-bottomed flask. The mixture was stirred and mixed to be uniform, and then heated to 30° C. After that, a reflux device was connected to the round-bottomed flask. 15 g of Novozym 435 was added thereto, and the resulting mixture was subjected to a reaction for 2 h with magnetic stirring at a rotation speed of 350 rpm, obtaining an immobilized lipase. The immobilized lipase was collected, and evaporated on a rotary evaporator to remove anhydrous ethanol. The composition of the resulting product was analyzed by liquid chromatography. The analysis result showed that the product had no PC, and only contained sn2-LPC and fatty acid ethyl ester, and the molar numbers of sn2-LPC and fatty acid ethyl ester were equal, and were equal to the initial molar number of PC, showing that PC was completely converted into sn2-LPC and fatty acid ethyl ester. The product was extracted with solvent (water and n-hexane in sequence) to separate sn2-LPC and fatty acid ethyl ester, obtaining an aqueous phase and an n-hexane phase. Cold acetone was added into the aqueous phase for depositing, obtaining sn2-LPC, and sn2-LPC was subjected to a methylesterification, and was then analyzed by gas chromatography to determine composition of fatty acid at sn-2 position of phosphatidylcholine. The n-hexane phase was subjected to a rotary evaporation to remove n-hexane, obtaining fatty acid ethyl ester. Fatty acid ethyl ester was analyzed by gas chromatography to determine composition of fatty acid at sn-1 position of phosphatidylcholine.

### Comparative Example 1

100 g of a mixture of soybean phosphatidylcholine and ethanol (95%) (a molar ratio of soybean phosphatidylcholine to 95% ethanol is 1:100) was added into a 500 mL round-bottomed flask. The mixture was stirred and mixed to be uniform, and then heated to 30° C. After that, a reflux device was connected to the round-bottomed flask. 15 g of Lipozyme RM IM was added thereto, and the resulting mixture was subjected to a reaction for 6 h with magnetic stirring at a rotation speed of 250 rpm, obtaining an immobilized lipase. The immobilized lipase was collected, and evaporated on a rotary evaporator to remove ethanol and water. The composition of the resulting product was analyzed by liquid chromatography. The analysis result showed that the product had 3.31 mol % of PC, showing that PC was not been completely converted into sn2-LPC and fatty acid ethyl ester.

### Comparative Example 2

100 g of a mixture of soybean phosphatidylcholine and ethanol (95%) (a molar ratio of soybean phosphatidylcholine to 95% ethanol being 1:100) was added into a 500 mL round-bottomed flask. The mixture was stirred and mixed to be uniform, and then heated to 30° C. After that, a reflux device was connected to the round-bottomed flask. 15 g of Lipozyme RM IM was added thereto, and the resulting mixture was subjected to a reaction for 8 h with magnetic stirring at a rotation speed of 250 rpm, obtaining an immobilized lipase. The immobilized lipase was collected, and evaporated on a rotary evaporator to remove ethanol and water. The composition of the resulting product was analyzed by liquid chromatography. The analysis result showed that the product had no PC, and contained sn2-LPC, fatty acid ethyl ester and fatty acid, and the sum of the molar number of fatty acid ethyl ester and fatty acid was slightly higher than that of sn2-LPC, and the sum of the molar number of sn2-LPC, fatty acid ethyl ester and fatty acid was 2 times the initial molar number of PC. The above results showed that there was a weak transfer of acyl during the reaction process. The product was extracted with solvent (water and n-hexane in sequence) to separate sn2-LPC, fatty acid ethyl ester and fatty acid, obtaining an aqueous phase and an n-hexane phase. Cold acetone was added into the aqueous phase for depositing, obtaining sn2-LPC, and sn2-LPC was subjected to a methylesterification, and was then analyzed by gas chromatography to determine composition of fatty acid at sn-2 position of phosphatidylcholine. The n-hexane phase was subjected to a rotary evaporation to remove n-hexane, obtaining fatty acid ethyl ester and fatty acid. Fatty acid ethyl ester and fatty acid were subjected to a methylesterification, and was then analyzed by gas chromatography to determine composition of fatty acid at sn-1 position of phosphatidylcholine. Compared with Examples 1-5, the accuracy of the analysis results in the comparative example was reduced due to the weak transfer of acyl group during the alcoholysis process. In addition, the reaction products contains a small amount of fatty acids, and thus fatty acid ethyl ester and fatty acid need to be analyzed by gas chromatography after the methylesterification of them, which increases the complexity of the analysis procedures. In short, the method in the comparative example provides low accuracy of the results and has a long reaction time and complex operation procedures.

What is claimed is:

1. A method for analysis of the positional distribution of fatty acid in phosphatidylcholine, comprising,
Step 1: mixing phosphatidylcholine and excess anhydrous ethanol to be uniform in a reaction vessel, to obtain a mixture, heating the mixture to a temperature for an alcoholysis reaction, and connecting a reflux device to the reaction vessel;
Step 2: adding immobilized lipase Novozym 435 or Lipozyme 435 into the reaction vessel, and subjecting the resulting mixture to the alcoholysis reaction while stirring, to obtain an alcoholysis product;
Step 3: extracting the alcoholysis product with water and n-hexane in sequence, to obtain an aqueous phase and an n-hexane phase, and collecting the aqueous phase and the n-hexane phase, respectively; adding cold acetone into the aqueous phase for depositing, to obtain sn2-LPC, and subjecting the n-hexane phase to a rotary evaporation to remove n-hexane, to obtain fatty acid ethyl ester; and
Step 4: directly analyzing the fatty acid ethyl ester by gas chromatography, and subjecting the sn2-LPC to a methylesterification, and analyzing the sn2-LPC after the methylesterification by gas chromatography.

2. The method as claimed in claim 1, wherein a molar ratio of phosphatidylcholine to anhydrous ethanol is in the range of 1:(40-100).

3. The method as claimed in claim 1, wherein in step 1, the temperature for an alcoholysis reaction is 25° C. to 40° C.

4. The method as claimed in claim 1, wherein in step 2, the immobilized lipase is added in an amount of 6%-15%, based on the total mass of a substrate.

5. The method as claimed in claim 1, wherein in step 2, the stirring is performed at a rotation speed of 250-500 rpm.

6. The method as claimed in claim 1, wherein the subjecting the resulting mixture to the alcoholysis reaction is performed for 1-3 h.
